(19) United States
(12) Patent Application Publication
(10) Pub. No.: US 2020/0255714 A1
(43) Pub. Date: Aug. 13, 2020

(54) HYDROFLUOROEPOXIDE CONTAINING COMPOSITIONS AND METHODS FOR USING SAME

(71) Applicant: 3M INNOVATIVE PROPERTIES COMPANY, St. Paul, MN (US)

(72) Inventors: Sean Michael Smith, Woodbury, MN (US); Karl Joseph Warren, Hudson, WI (US); Zhongxing Zhang, Woodbury, MN (US)

(21) Appl. No.: 16/755,896

(22) PCT Filed: Oct. 22, 2018
(86) PCT No.: PCT/IB2018/058203

§ 371 Date: Apr. 14, 2020

Related U.S. Application Data
(60) Provisional application No. 62/576,110, filed on Oct. 24, 2017.

(51) Int. Cl. C09K 5/10; C07D 303/08; B23K 1/015

(52) U.S. Cl. CPC C09K 5/10; B23K 1/015; C07D 303/08

(57) ABSTRACT

A composition includes a hydrofluoroepoxide having Structural Formula (I). Each R_{f} is, independently, a linear or branched perfluoroalkyl group having 1-6 carbon atoms and optionally comprises a catenated heteroatom.

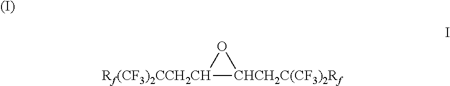

### FIELD

This disclosure relates to compositions and devices that include hydrofluoroepoxides, and methods of making and using same.

### BACKGROUND

Various hydrofluoroepoxides are described in, for example, U.S. Pat. Nos. 6,180,113, 5,101,058, and 7,226,578.

### SUMMARY

In some embodiments, a hydrofluoroepoxide having Structural Formula (I) is provided.

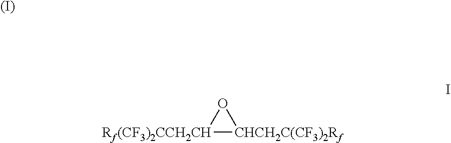

Each R_{f} is, independently, a linear or branched perfluoroalkyl group having 1-6 carbon atoms and optionally comprises a catenated heteroatom.

The above summary of the present disclosure is not intended to describe each embodiment of the present disclosure. The details of one or more embodiments of the disclosure are also set forth in the description below. Other features, objects, and advantages of the disclosure will be apparent from the description and from the claims.

### DETAILED DESCRIPTION

The development of inert fluorinated fluids that have relatively short atmospheric lifetimes and low global warming potentials, while providing high thermal stability, low toxicity, good solvency, and a wide operating temperature to meet various application requirements are of particular interest. Currently, high boiling materials (e.g., >220° C.) used in industry primarily consist of perfluorinated inerts which are high in environmental persistence and global warming potential. Consequently, the development of more environmentally benign materials which also exhibit high thermal stability, thermal conductivity, and chemical inertness at high operating temperatures is desirable.

Generally, the present disclosure relates to fluorinated epoxide-containing hydrofluorocarbons, or hydrofluoroepoxides, and the method of their synthesis. The hydrofluoroepoxides promote facile atmospheric degradation resulting in relatively short atmospheric lifetimes, particularly when compared to perfluorinated hydrocarbons (PFCs) and hydrofluorocarbons (HFCs). Furthermore, despite short atmospheric lifetimes, the compounds are stable at elevated temperatures (e.g., >220° C.) and resistant towards further oxidation under oxidative conditions.

In this disclosure:

“device” refers to an object or contrivance which is heated, cooled, or maintained at a predetermined temperature or temperature range;

“inert” refers to chemical compositions that are generally not chemically reactive under normal conditions of use;

“mechanism” refers to a system of parts or a mechanical appliance;

“perfluoro-” (for example, in reference to a group or moiety, such as in the case of “perfluoroalkylene” or “perfluoroalkylcarbonyl” or “perfluorinated”) means completely fluorinated such that, except as may be otherwise indicated, there are no carbon-bonded hydrogen atoms replaceable with fluorine; and

“catenated heteroatom” means an atom other than carbon (for example, oxygen, nitrogen, or sulfur) that is bonded to at least two carbon atoms in a carbon chain (linear or branched or within a ring) so as to form a carbon-heteroatom-carbon linkage.

As used herein, the singular forms “a”, “an”, and “the” include plural referents unless the content clearly dictates otherwise. As used in this specification and the appended embodiments, the term “or” is generally employed in its sense including “and/or” unless the content clearly dictates otherwise.

As used herein, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

In some embodiments, the compositions of the present disclosure may include one or more hydrofluoroepoxides having Structural Formula (I):

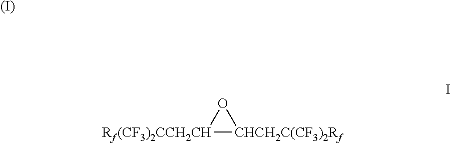

In some embodiments, each R_{f} may be, independently, a linear or branched perfluoroalkyl group having 1-6, 1-4, or 1-3 carbon atoms and optionally includes one or more catenated heteroatoms (e.g., oxygen or nitrogen heteroatoms). In some embodiments, each R_{f} group may be the same linear or branched perfluoroalkyl group. It is to be recognized that the hydrofluoroepoxides of the present disclosure may include the cis isomer, the trans isomer, or a mixture of the cis and trans isomers, irrespective of what is depicted in any of the general formulas or chemical structures.

In various embodiments, representative examples of the hydrofluoroepoxides of general formula (I) include the following:

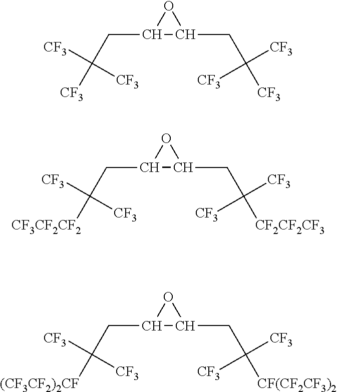

The hydrofluoroepoxides of the present disclosure have been discovered to possess short atmospheric lifetimes and low global warming potentials, while providing low toxicity, adequate solvency, and high thermal stability. Further regarding the high thermal stability of the hydrofluoroepoxides, it has been discovered that the presence of a quaternary carbon at a position adjacent the methylene group that is adjacent the epoxide carbon enabled such high temperature stability. Specifically, it was discovered that similar epoxides not having such a quaternary carbon resulted in dehydrofluorination (HF generation) at elevated temperatures which, in turn, is associated with undesirable corrosion and safety issues.

In some embodiments, the present disclosure is directed to a working fluid that includes one or more of the above-described hydrofluoroepoxides. For example, the working fluids may include at least 25%, at least 50%, at least 70%, at least 80%, at least 90%, at least 95%, or at least 99% by weight of the above-described hydrofluoroepoxides based on the total weight of the working fluid. In addition to the above-described hydrofluoroepoxides, the working fluids may include a between 0.1 and 75%, between 0.1 and 50%, between 0.1 and 30%, between 0.1 and 20%, between 0.1 and 10%, between 0.1 and 5%, or between 0.1 and 1% by weight of one or more of the following components (individually or in any combination): alcohols, ethers, alkanes, alkenes, perfluorocarbons, perfluorinated tertiary amines, perfluoroethers, cycloalkanes, esters, ketones, oxiranes, aromatics, siloxanes, hydrochlorocarbons, hydrochlorofluorocarbons, hydrofluorocarbons, hydrofluoroolefins, hydrochlorofluoroolefins, hydrofluoroethers, perfluoroketones, or mixtures thereof, based on the total weight of the working fluid. Such additional components can be chosen to modify or enhance the properties of a composition for a particular use. Minor amounts of optional components can also be added to the working fluids to impart particular desired properties for particular uses. Useful components can include conventional additives such as, for example, surfactants, coloring agents, stabilizers, anti-oxidants, flame retardants, and the like, and mixtures thereof.

In some embodiments, the working fluids of the present disclosure may exhibit properties that render them particularly useful as heat transfer fluids. For example, the working fluids may be chemically inert (i.e., they do not easily react with base, acid, water, etc.), and may have high boiling points (up to 300° C.), low freezing points (they may be liquid at −40° C. or lower), low viscosity, high thermal stability over extended periods, good thermal conductivity, adequate solvency in a range of potentially useful solvents, and low toxicity.

Hydrocarbon alkenes are known to react with hydroxyl radicals and ozone in the lower atmosphere at rates sufficient to lead to short atmospheric lifetimes (see Atkinson, R.; Arey, J., Chem Rev. 2003, 103 4605-4638). For example, ethene has an atmospheric lifetime by reaction with hydroxyl radicals and ozone of 1.4 days and 10 days, respectively. Propene has an atmospheric lifetime by reaction with hydroxyl radicals and ozone of 5.3 hours and 1.6 days, respectively. Both the E and Z isomers of the hydrofluoroolefins of the present disclosure were found to react at a very high rate with ozone in the gas phase. As a result, it is believed that these compounds have relatively short atmospheric lifetimes.

Furthermore, in some embodiments, the working fluids of the present disclosure may have a low environmental impact. In this regard, the working fluids may have a global warming potential (GWP) of less 300, 200, or even less than 100. As used herein, GWP is a relative measure of the warming potential of a compound based on the structure of the compound. The GWP of a compound, as defined by the

Intergovernmental Panel on Climate Change (IPCC) in 1990 and updated in 2007, is calculated as the warming due to the release of 1 kilogram of a compound relative to the warming due to the release of 1 kilogram of CO2 over a specified integration time horizon (ITH).

$${GWP}_{i} ( {t}^{′} ) = \frac{{{\int }^{ITH}}_{0} {a}_{i} [ C ( t ) ] dt}{{{\int }^{ITH}}_{0} {a}_{{co}_{i}} [ {C}_{{co}_{2}} ( t ) ] dt} = \frac{{{\int }^{ITH}}_{0} {a}_{i} {C}_{o ɛ} {e}^{- t / \tilde{a}} dt}{{{\int }^{ITH}}_{0} {a}_{{co}_{3}} [ {C}_{{co}_{2}} ( t ) ] dt}$$

In this equation aᵢ is the radiative forcing per unit mass increase of a compound in the atmosphere (the change in the flux of radiation through the atmosphere due to the IR absorbance of that compound), C is the atmospheric concentration of a compound, τ is the atmospheric lifetime of a compound, t is time, and i is the compound of interest. The commonly accepted ITH is 100 years representing a compromise between short-term effects (20 years) and longer-term effects (500 years or longer). The concentration of an organic compound, i, in the atmosphere is assumed to follow pseudo first order kinetics (i.e., exponential decay). The concentration of CO2 over that same time interval incorporates a more complex model for the exchange and removal of CO2 from the atmosphere (the Bern carbon cycle model).

In some embodiments, the hydrofluoroepoxides having Structural Formula (I) may be synthesized in high yield via the allylic halide substitution/olefin oxidation sequence illustrated in Scheme 1.

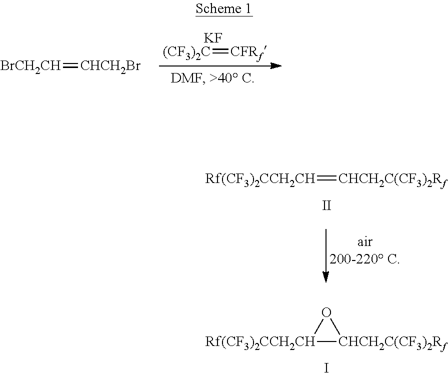

The first step of Scheme 1 may involve the substitution of a 1,4-dibromo-2-butene by an activated perfluorinated olefin nucleophile which is formed in situ by contacting perfluorinated olefin (CF3)₂C═CFR_{f}′ with KF. The second step of Scheme 1 (i.e., the epoxidation of II to result in I) may be carried out in a metal pressure reactor. Compound II may be sealed in the metal reactor and the inside may then be pressurized with air (as high as 88 psi). With agitation, the contents of the sealed reactor may then be heated (>200° C.) to effectively oxidize the olefin starting material to afford compound I. The process may be repeated several times until complete conversion of compound II. Purification by fractional distillation under reduced pressure may yield the desired epoxide product.

The working fluids of the present disclosure can be used in various applications. For example, the working fluids are believed to possess the required stability as well as the necessary short atmospheric lifetime (or low global warming potential) to make them commercially viable environmentally-friendly candidates for high temperature heat transfer applications.

In some embodiments, the present disclosure is further directed to an apparatus for heat transfer that includes a device and a mechanism for transferring heat to or from the device. The mechanism for transferring heat may include a heat transfer fluid that includes the working fluids of the present disclosure.

The provided apparatus for heat transfer may include a device. The device may be a component, work-piece, assembly, etc. to be cooled, heated or maintained at a predetermined temperature or temperature range. Such devices include electrical components, mechanical components and optical components. Examples of devices of the present disclosure include, but are not limited to microprocessors, wafers used to manufacture semiconductor devices, power control semiconductors, electrical distribution switch gear, power transformers, circuit boards, multi-chip modules, packaged and unpackaged semiconductor devices, lasers, chemical reactors, fuel cells, and electrochemical cells. In some embodiments, the device can include a chiller, a heater, or a combination thereof.

In yet other embodiments, the devices can include electronic devices, such as processors, including microprocessors. As these electronic devices become more powerful, the amount of heat generated per unit time increases. Therefore, the mechanism of heat transfer plays an important role in processor performance. The heat-transfer fluid typically has good heat transfer performance, good electrical compatibility (even if used in “indirect contact” applications such as those employing cold plates), as well as low toxicity, low (or non-) flammability and low environmental impact. Good electrical compatibility suggests that the heat-transfer fluid candidate exhibit high dielectric strength, high volume resistivity, and poor solvency for polar materials. Additionally, the heat-transfer fluid should exhibit good mechanical compatibility, that is, it should not affect typical materials of construction in an adverse manner.

The provided apparatus may include a mechanism for transferring heat. The mechanism may include a heat transfer fluid. The heat transfer fluid may include the working fluids of the present disclosure. Heat may be transferred by placing the heat transfer mechanism in thermal contact with the device. The heat transfer mechanism, when placed in thermal contact with the device, removes heat from the device or provides heat to the device, or maintains the device at a selected temperature or temperature range.

The direction of heat flow (from device or to device) is determined by the relative temperature difference between the device and the heat transfer mechanism.

The heat transfer mechanism may include facilities for managing the heat-transfer fluid, including, but not limited to pumps, valves, fluid containment systems, pressure control systems, condensers, heat exchangers, heat sources, heat sinks, refrigeration systems, active temperature control systems, and passive temperature control systems.

Examples of suitable heat transfer mechanisms include, but are not limited to, temperature controlled wafer chucks in plasma enhanced chemical vapor deposition (PECVD) tools, temperature-controlled test heads for die performance testing, temperature-controlled work zones within semiconductor process equipment, thermal shock test bath liquid reservoirs, and constant temperature baths. In some systems, such as etchers, ashers, PECVD chambers, vapor phase soldering devices, and thermal shock testers, the upper desired operating temperature may be as high as 170° C., as high as 200° C., or even as high as 240° C.

Heat can be transferred by placing the heat transfer mechanism in thermal contact with the device. The heat transfer mechanism, when placed in thermal contact with the device, may remove heat from the device or provide heat to the device, or maintain the device at a selected temperature or temperature range. The direction of heat flow (from device or to device) is determined by the relative temperature difference between the device and the heat transfer mechanism. The provided apparatus can also include refrigeration systems, cooling systems, testing equipment and machining equipment. In some embodiments, the provided apparatus can be a constant temperature bath or a thermal shock test bath. In some systems, such as etchers, ashers, PECVD chambers, vapor phase soldering devices, and thermal shock testers, the upper desired operating temperature may be as high as 170° C., as high as 200° C., or even higher.

In some embodiments, the working fluids of the present disclosure may be used as a heat transfer agent for use in vapor phase soldering. In using the working fluids of the present disclosure in vapor phase soldering, the process described in, for example, U.S. Pat. No. 5,104,034 (Hansen) can be used, which description is hereby incorporated by reference in its entirety. Briefly, such process includes immersing a component to be soldered in a body of vapor comprising the working fluids of the present disclosure to melt the solder. In carrying out such a process, a liquid pool of the working fluid is heated to boiling in a tank to form a saturated vapor in the space between the boiling liquid and a condensing means.

A workpiece to be soldered is immersed in the vapor (at a temperature of greater than 170° C., greater than 200° C., greater than 230° C., or even greater), whereby the vapor is condensed on the surface of the workpiece so as to melt and reflow the solder. Finally, the soldered workpiece is then removed from the space containing the vapor.

### Listing of Embodiments

1. A composition comprising:

a hydrofluoroepoxide having Structural Formula (I):

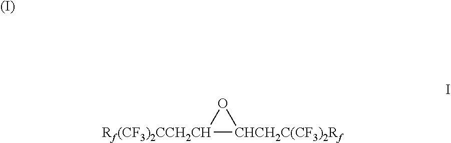

wherein each R_{f} is, independently, a linear or branched perfluoroalkyl group having 1-6 carbon atoms and optionally comprises a catenated heteroatom.
2. The composition of embodiment 1, wherein each R_{f} is the same linear or branched perfluoroalkyl group.
3. The composition of embodiment 1, wherein the hydrofluoroepoxide comprises one or more of the following hydrofluoroepoxides:

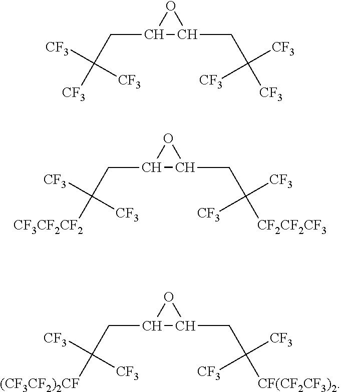

4. The composition according to any one of the previous embodiments, wherein the hydrofluoroepoxide is present in the composition in an amount of at least 50% by weight based on the total weight of the composition.
5. An apparatus for heat transfer comprising:

a device; and

a mechanism for transferring heat to or from the device, the mechanism comprising a heat transfer fluid that comprises the composition according to any one of the previous embodiments.
6. An apparatus for heat transfer according to embodiment 5, wherein the device is selected from a microprocessor, a semiconductor wafer used to manufacture a semiconductor device, a power control semiconductor, an electrochemical cell, an electrical distribution switch gear, a power transformer, a circuit board, a multi-chip module, a packaged or unpackaged semiconductor device, a fuel cell, and a laser.
7. An apparatus according to embodiment 5, wherein the mechanism for transferring heat is a component in a system for maintaining a temperature or temperature range of an electronic device.
8. An apparatus according to embodiment 5, wherein the device comprises an electronic component to be soldered.
9. An apparatus according to embodiment 5, wherein the mechanism comprises vapor phase soldering.
10. A method of transferring heat comprising:

providing a device; and

transferring heat to or from the device using the composition of any one of embodiments 1-4.

Objects and advantages of this disclosure are further illustrated by the following examples, but the particular materials and amounts thereof recited in these examples, as well as other conditions and details, should not be construed to unduly limit this disclosure.

### EXAMPLES

Objects and advantages of this disclosure are further illustrated by the following comparative and illustrative examples. Unless otherwise noted, all parts, percentages, ratios, etc. in the examples and the rest of the specification are by weight, and all reagents used in the examples were obtained, or are available, from general chemical suppliers such as, for example, Sigma-Aldrich Corp., Saint Louis, Mo., US or Alfa Aesar, Haverhill, Mass., US, or may be synthesized by conventional methods.

The following abbreviations are used in this section: mL=milliliter, min=minutes, h=hours, g=gram, μm=micron, mmol=millimole, ° C.=degrees Celsius.

| Material | Source |
|---|---|
| trans-1,4-dibromo-2-butene | Sigma-Aldrich Corp., Saint Louis, |
| | MO, US |
| cis-1,4-dichloro-2-butene | Sigma-Aldrich Corp., Saint Louis, |
| | MO, US |
| Methyltrialkyl (C8-C10) | Sigma-Aldrich Corp., Saint Louis, |
| ammonium chloride | MO, US |
| (Adogen 464) | |
| Potassium Fluoride, | SAFC Commercial Life Science |
| spray dried (KF) | Products and Services, WI, US |
| Hexafluoropropene (HFP) dimer | Alfa Aesar, Haverhill, MA, US |
| Potassium iodide (KI) | Alfa Aesar, Haverhill, MA, US |
| N,N-Dimethylformamide (DMF) | Sigma-Aldrich Corp., Saint Louis, |
| | MO, US |
| Tetraethylene glycol dimethyl | Alfa Aesar, Haverhill, MA, US |
| ether (tetraglyme) | |
| Dichloromethane (DCM) | Sigma-Aldrich Corp., Saint Louis, |
| | MO, US |
| Fluorinated ethylene propylene | Sigma-Aldrich Corp., Saint Louis, |
| (FEP) tubing, 1/4 inch (6.35 mm) | MO, US |
| and 1/8 inch (3.18 mm) diameter | |
| 3-Chloroperbenzoic acid | Sigma-Aldrich Corp., Saint Louis, |
| (MCPBA) | MO, US |
| Molybdenum hexacarbonyl | Sigma-Aldrich Corp., Saint Louis, |
| (Mo(CO)₆) | MO, US |
| 4 Angstrom (Å) molecular sieves | Sigma-Aldrich Corp., Saint Louis, |
| | MO, US |
| Basic alumina | Alfa Aesar, Haverhill, MA, US |
| Silica gel | Alfa Aesar, Haverhill, MA, US |
| Activated carbon | Alfa Aesar, Haverhill, MA, US |
| Potassium carbonate | Alfa Aesar, Haverhill, MA, US |
| N-hydroxyphthalimide | Alfa Aesar, Haverhill, MA, US |
| Ethylbenzene | Alfa Aesar, Haverhill, MA, US |

Three sets of conditions were used for the oxidation of hydrofluoroolefin to afford the respective hydrofluoroepoxide product. Method A utilized a 600 mL stainless steel Parr reaction vessel charged with hydrofluoroolefin and pressurized by air. Method B utilized a 500 mL 3-neck round bottom flask equipped with a temperature probe, magnetic stir bar, water-cooled condenser, and a ¼ inch FEP tube for sparging with air. Method C utilized a 500 mL 3- or 4-neck round bottom flask equipped with a temperature probe, magnetic stir bar, water-cooled condenser, and one or two ⅛ inch FEP tube(s) with one or two 10 micron steel frit(s).

### Preparatory Example PE1: Synthesis of 2,3-bis(3,3,4,4,5,5,5-heptafluoro-2,2-bis(trifluoromethyl)pentyl)oxirane from (E)-1,1,1,2,2,3,3,10,10,11,11,12,12,12-tetradecafluoro-4,4,9,9-tetrakis(trifluoromethyl)dodec-6-ene

(E)-1,1,1,2,2,3,3,10,10,11,11,12,12,12-tetradecafluoro-4,4,9,9-tetrakis(trifluoromethyl)dodec-6-ene was prepared from the substitution of 1,4-dibromo-2-butene by HFP dimer in a mixture of Adogen 464, KF, and DMF as described in PCT Application Publication WO16094113.

Method A: To a 600 mL stainless steel Parr reaction vessel was added (E)-1,1,1,2,2,3,3,10,10,11,11,12,12,12-tetradecafluoro-4,4,9,9-tetrakis(trifluoromethyl)dodec-6-ene. The reactor was sealed and then pressurized by the addition of air (50 psi, 345 kPa). The internal temperature was slowly raised to 248° C. with stirring and the pressure reached 74 psi (510 kPa). After a 16 hour stir, the internal temperature was allowed to cool to a temperature followed by venting and a recharge of air to an internal pressure of 43 psi (296 kPa). With stirring, the internal temperature was reheated to 250° C. and the internal pressure of the reaction vessel had reached 88 psi (607 kPa). The reaction material was allowed to stir for 16 hours at the same temperature and was re-cooled to room temperature. The vessel was once again vented and then recharged with air (43 psi, 296 kPa), heated with stirring (250° C.), allowed to stir for 48 hours, cooled to room temperature followed by venting to complete the third run. Runs 4-8 were completed under the following conditions: Run 4 (52 psi (359 kPa), 250° C., 6 h stir); Run 5 (52 psi (359 kPa), 250° C., 16 h); Run 6 (70 psi (483 kPa), 250 C, 16 h); Run 7 (80 psi (552 kPa), 250° C., 16 h); Run 8 (80 psi (552 kPa), 250° C., 16 h). After the final run, 90 g of crude reaction material was obtained for which GC analysis indicated 67% conversion of the hydrofluoroolefin starting material. GC analysis also indicated 41% of the reaction mixture consisted of the desired oxidation product, 2,3-bis(3,3,4,4,5,5,5-heptafluoro-2,2-bis(trifluoromethyl)pentyl) oxirane. GC-MS analysis coupled with ¹⁹F and ¹H NMR confirmed the structure to be that of the desired product.

Method B: To a 500 mL 3-neck round-bottom flask equipped with a stir bar, temperature probe, ¼ inch FEP tube, and a water-cooled condenser was added (E)-1,1,1,2,2,3,3,10,10,11,11,12,12,12-tetradecafluoro-4,4,9,9-tetrakis(trifluoromethyl)dodec-6-ene (200.1 g, 289 mmol). With stirring, the starting material was slowly heated to 211.5° C. while sparging air through a ¼ inch FEP tube. After an 84 hour stir at a temperature range of 211.5° C.-220° C., the resultant mixture was allowed to cool to room temperature. The resultant 145 g of crude reaction material contained 85% of the desired epoxide material. The reaction product was purified by concentric tube distillation under reduced pressure (113° C., 3 Torr) to afford 2,3-bis(3,3,4,4,5,5,5-heptafluoro-2,2-bis(trifluoromethyl)pentyl) oxirane (123 g, 60% yield). The desired 2,3-bis(3,3,4,4,5,5,5-heptafluoro-2,2-bis(trifluoromethyl)pentyl)oxirane composition was confirmed by GC-MS analysis coupled with ¹H and ¹⁹F NMR spectroscopy.

Method C: To a 500 mL 4-neck round-bottom flask equipped with a stir bar, temperature probe, two ⅛ inch FEP tubes connected to 10 micron steel frits, and a water-cooled condenser was added (E)-1,1,1,2,2,3,3,10,10,11,11,12,12,12-tetradecafluoro-4,4,9,9-tetrakis(trifluoromethyl)dodec-6-ene (206 g, 298 mmol). The internal temperature was raised to 209° C. with stirring and sparging by air through the two 10 micron steel frits commenced (0.4 L/min). After stirring for 135 hours with the internal temperature held between 206-209° C., the reaction was allowed to cool to room temperature and sparging by air was ceased to afford 156 g of a light yellow liquid. Analysis of the crude reaction mixture by GC revealed >70% conversion of the hydrofluoroolefin starting material and that 60% of the mixture consisted of the desired hydrofluoroepoxide 2,3-bis(3,3,4,4,5,5,5-heptafluoro-2,2-bis(trifluoromethyl)pentyl)oxirane (93.6 g, 44% yield).

### Preparatory Example 2: Synthesis of 2,3-bis(3,3,4,4,5,5,5-heptafluoro-2,2-bis(trifluoromethyl)pentyl)oxirane from (Z)-1,1,1,2,2,3,3,10,10,11,11,12,12,12-tetradecafluoro-4,4,9,9-tetrakis(trifluoromethyl)dodec-6-ene

(Z)-1,1,1,2,2,3,3,10,10,11,11,12,12,12-tetradecafluoro-4,4,9,9-tetrakis(trifluoromethyl)dodec-6-ene was prepared from the substitution of cis-1,4-dichloro-2-butene by HFP dimer in a mixture of Adogen 464, KF, and DMF as described in PCT Application Publication WO16094113.

Method B: To a 500 mL 3-neck round-bottom flask equipped with a stir bar, temperature probe, ¼ inch FEP tube, and a water-cooled condenser was added (Z)-1,1,1,2,2,3,3,10,10,11,11,12,12,12-tetradecafluoro-4,4,9,9-tetrakis(trifluoromethyl)dodec-6-ene (340 g, 501 mmol). The internal temperature was raised to 215° C. with stirring and sparging by air through the ¼ inch FEP tube. After an 88 hour stir with the internal temperature held at 215° C., the reaction was allowed to cool to room temperature and sparging by air was ceased to afford a light yellow liquid. Analysis of the crude reaction material by GC revealed >92% conversion of the hydrofluoroolefin starting material and that 78% of the mixture consisted of the desired hydrofluoroepoxide. Purification via concentric tube distillation under reduced pressure (113° C., 3 torr) afforded 2,3-bis(3,3,4,4,5,5,5-heptafluoro-2,2-bis(trifluoromethyl)pentyl)oxirane (195 g, 55% yield) as a light yellow liquid.

Method C: To a 500 mL 4-neck round-bottom flask equipped with a stir bar, temperature probe, two ⅛ inch FEP tubes connected to 10 micron steel frits, and a water-cooled condenser was added (Z)-1,1,1,2,2,3,3,10,10,11,11,12,12,12-tetradecafluoro-4,4,9,9-tetrakis(trifluoromethyl)dodec-6-ene (202 g, 292 mmol). The internal temperature was raised to 205° C. with stirring and sparging by air through the two 10 micron steel frits commenced (0.4 L/min). After an 87 hour stir with the internal temperature held between 205-212° C., the reaction was allowed to cool to room temperature and sparging by air was ceased to afford 159 g of a light yellow liquid. Analysis of the crude reaction mixture by GC revealed >56% conversion of the hydrofluoroolefin starting material and that 48% of the mixture consisted of the desired hydrofluoroepoxide 2,3-bis(3,3,4,4,5,5,5-heptafluoro-2,2-bis(trifluoromethyl)pentyl) oxirane (76 g, 37% yield).

### Comparative Example CE1. Attempted MCPBA Oxidation of (E)-1,1,1,2,2,3,3,10,10,11,11,12,12,12-tetradecafluoro-4,4,9,9-tetrakis(trifluoromethyl)dodec-6-ene

Method A: To a two-neck flask equipped with a water-cooled condenser and magnetic stir bar were added dichloromethane (DCM, 50 mL) and (E)-1,1,1,2,2,3,3,10,10,11,11,12,12,12-tetradecafluoro-4,4,9,9-tetrakis(trifluoromethyl)dodec-6-ene (30 g, 43 mmol). The resultant mixture was allowed to cool to 0° C. To the resultant mixture was slowly added 3-chloroperoxybenzoic acid (MCPBA, 20.2 g of 50% in water, 59 mmol) followed by a 12 h stir at the same temperature. GC-FID analysis of the crude reaction material revealed only starting material and no peaks indicating oxidation products.

Method B: To a two-neck flask equipped with a water-cooled condenser and magnetic stir bar were added dichloromethane (DCM), 50 mL) and (E)-1,1,1,2,2,3,3,10,10,11,11,12,12,12-tetradecafluoro-4,4,9,9-tetrakis(trifluoromethyl)dodec-6-ene (30 g, 43 mmol). With stirring at room temperature, 3-chloroperoxybenzoic acid (MCPBA, 20.2 g of 50% in water, 59 mmol) was added dropwise and the resultant mixture was slowly heated to reflux followed by a 12 h stir. GC-FID analysis of the crude reaction material revealed only starting material and no peaks indicating oxidation products.

### Comparative Example CE2. Mo(CO)₆-Catalyzed Oxidation of (Z)-1,1,1,2,2,3,3,10,10,11,11,12,12,12-tetradecafluoro-4,4,9,9-tetrakis(trifluoromethyl)dodec-6-ene

To a 3-neck round bottom flask equipped with a water-cooled reflux condenser, temperature probe, and stir bar were added (Z)-1,1,1,2,2,3,3,10,10,11,11,12,12,12-tetradecafluoro-4,4,9,9-tetrakis(trifluoromethyl)dodec-6-ene (30 g, 43 mmol), molybdenumhexacarbonyl (1.2 g, 4.3 mmol), N-hydroxyphthalimide (0.71 g, 4.3 mmol), and ethylbenzene (5.4 g, 51 mmol). The mixture was stirred and then charged with oxygen gas and the reflux condenser was equipped with a balloon to maintain the oxygen gas atmosphere throughout the reaction. The mixture was slowly heated to 100° C. and was allowed to stir overnight. The resultant mixture was then analyzed by GC-FID and no peaks indicating oxidation products were observed.

### Application Example AE1: Thermal Stability of 2,3-bis(3,3,4,4,5,5,5-heptafluoro-2,2-bis(trifluoromethyl)pentyl)oxirane

To a 3-neck 250 mL round bottom flask equipped with a water-cooled condenser, temperature probe, magnetic stir bar, and a ⅛ inch FEP tube connected to 10 micron steel frit was charged 2,3-bis(3,3,4,4,5,5,5-heptafluoro-2,2-bis(trifluoromethyl)pentyl) oxirane (70.4 g, 99 mmol). Sparging by air through the 10 micron steel frit at a rate of 0.4 L/min commenced and the internal temperature was slowly raised to 220° C. with stirring. After a 72 hour stir with the temperature held between 215° C.-220° C., the material was allowed to cool to room temperature. The resultant material was weighed (70.3 g, 99 mmol) and GC analysis revealed no decomposition with the final mixture containing approximately 99.4% of the 2,3-bis(3,3,4,4,5,5,5-heptafluoro-2,2-bis(trifluoromethyl)pentyl)oxirane starting material.

### Application Example (AE2): Chemical Stability of 2,3-bis(3,3,4,4,5,5,5-heptafluoro-2,2-bis(trifluoromethyl)pentyl)oxirane at Elevated Temperature

The chemical stability was evaluated by charging a weighed amount of Preparatory Example PE1 into glass vials and then adding a weighed amount of absorbent. The samples were stirred with heating at 65° C. for 16 hours and then analyzed by GC-FID to determine whether any breakdown products were formed and whether the level of purity changed. The test results using various absorbents are shown in Table 1. This data indicates that the material can be useful for heat transfer and vapor phase soldering applications because it demonstrates stability in the presence of various absorbents at elevated temperatures.

TABLE 1
Chemical Stability of 2,3-bis(3,3,4,4,5,5,5-heptafluoro-
2,2-bis(trifluoromethyl)pentyl)oxirane (PE1) at 65° C.

| | Initial | | 4 Å | | | |
|---|---|---|---|---|---|---|
| | starting | Activated | molecular | Basic | Silica | Potassium |
| | material | carbon | sieves | alumina | gel | carbonate |
| GC-FID | 99.6 | 98.4 | 99.6 | 99.7 | 99.6 | 99.6 |
| purity (%) | | | | | | |

### Application Example (AE3): Vapor Pressure of 2,3-bis(3,3,4,4,5,5,5-heptafluoro-2,2-bis(trifluoromethyl)pentyl)oxirane

Vapor Pressure was measured using the stirred-flask ebuilliometer method described in ASTM E 1719-97 “Vapor Pressure Measurement by Ebuilliometry.” This method is also referred to as “Dynamic Reflux.” The method uses a 50-mL glass round-bottom flask. Vacuum was measured and controlled using a J-KEM vacuum controller (J-KEM Scientific, Saint Louis, Mo., US). The pressure transducer was calibrated on the day of measurement by comparison with full vacuum and with an electronic barometer. The procedure was to begin slowly heating the material, then vacuum was applied until boiling occurred and a steady drop wise reflux rate was established. Pot temperature and pressure reading were recorded, then the vacuum controller was set for a higher absolute pressure and the material was heated further until a new reflux point was established. The pressure level was raised in increments until the vapor pressure curve was obtained up to the atmospheric boiling point. Vapor pressures of Preparatory Example 1 (PE1) are shown in Table 2. The boiling point of PE1 at 760 mmHg was 238.3° C. This vapor pressure data indicate that this material would be useful in heat transfer and vapor phase soldering applications.

TABLE 2
Vapor pressure of 2,3-bis(3,3,4,4,5,5,5-heptafluoro-2,2-bis
(trifluoromethyl)pentyl)oxirane (PE1) at Various Temperatures

| | Vapor pressure, |
|---|---|
| Temperature, ° C. | mmHg |
| 20 | 0.026 |
| 55 | 0.35 |
| 78.6 | 1.5 |
| 170.7 | 96.6 |
| 204.3 | 296.9 |
| 214.7 | 397.1 |
| 237.7 | 731.3 |
| 238.3 | Boiling Point |

### Application Example (AE4): Kinematic Viscosity of 2,3-bis(3,3,4,4,5,5,5-heptafluoro-2,2-bis(trifluoromethyl)pentyl)oxirane

Kinematic Viscosity was measured using glass SCHOTT Ubbelohde capillary viscometers (Xylem, Inc., Germany). The viscometers were timed using using a SCHOTT AVS 350 viscosity timer. For a temperature of 10° C., a Lawler temperature control bath was used (Lawler Manufacturing Corp., Edison, N.J., US). The viscometer measurement stand and glass viscometer were immersed in a temperature-controlled liquid bath filled with NOVEC 7500 (3M Company, Saint Paul, Minn., US) as the bath fluid. The Lawler temperature bath was fitted with a copper tubing coil for liquid nitrogen cooling with fine temperature control provided by the bath's electronic temperature control heater. The fluid was mechanically stirred to provide uniform temperature in the bath. The bath controlled temperature to within ±0.1° C., as measured by the built-in resistance temperature detector (RTD) temperature sensor. The sample liquid was added to the viscometer between the two fill lines etched on the viscometer. The AVS-350 automatically pumped the sample fluid above the upper timing mark, then released the fluid and measured the efflux times between the upper and lower timing marks. The fluid meniscus was detected by optical sensors as it passed each timing mark. The sample was drawn up and measured repeatedly, averaging multiple measurements. The glass viscometers were calibrated using Canon certified kinematic viscosity standard fluids to obtain a calibration constant (centistokes per second) for each viscometer. The measured kinematic viscosity (centistokes), was calculated as the average efflux time (seconds) times the constant (centistokes/second) for the viscometer used. Table 3 summarizes the results for Preparatory Example 1 (PE1) is shown in. This data demonstrates that the material has favorable viscosity at higher temperatures which enables its use as a fluid for heat transfer and vapor phase soldering applications.

TABLE 3
Kinematic Viscosity of 2,3-bis(3,3,4,4,5,5,5-heptafluoro-
2,2-bis(trifluoromethyl)pentyl)oxirane (PE1)

| | Kinematic Viscosity |
|---|---|
| Temperature, ° C. | (centistokes, cSt) |
| 10.0 | 146.42 |
| 25.0 | 36.22 |
| 30.0 | 25.87 |
| 40.0 | 14.49 |

Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows. All references cited in this disclosure are herein incorporated by reference in their entirety.

What is claimed is:

1. A composition comprising:
a hydrofluoroepoxide having Structural Formula (I): 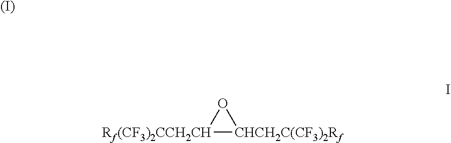
wherein each R_{f} is, independently, a linear or branched perfluoroalkyl group having 1-6 carbon atoms and optionally comprises a catenated heteroatom.

2. The composition of claim 1, wherein each R_{f} is the same linear or branched perfluoroalkyl group.

3. The composition of claim 1, wherein the hydrofluoroepoxide comprises one or more of the following hydrofluoroepoxides:

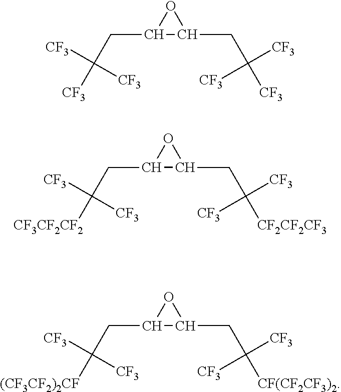

4. The composition according to claim 1, wherein the hydrofluoroepoxide is present in the composition in an amount of at least 50% by weight based on the total weight of the composition.

5. An apparatus for heat transfer comprising:
a device; and
a mechanism for transferring heat to or from the device, the mechanism comprising a heat transfer fluid that comprises the composition according to claim 1.

6. An apparatus for heat transfer according to claim 5, wherein the device is selected from a microprocessor, a semiconductor wafer used to manufacture a semiconductor device, a power control semiconductor, an electrochemical cell, an electrical distribution switch gear, a power transformer, a circuit board, a multi-chip module, a packaged or unpackaged semiconductor device, a fuel cell, and a laser.

7. An apparatus according to claim 5, wherein the mechanism for transferring heat is a component in a system for maintaining a temperature or temperature range of an electronic device.

8. An apparatus according to claim 5, wherein the device comprises an electronic component to be soldered.

9. An apparatus according to claim 5, wherein the mechanism comprises vapor phase soldering.

10. A method of transferring heat comprising:
providing a device; and
transferring heat to or from the device using the composition of claim 1.
